(19) United States
(12) Patent Application Publication
(10) Pub. No.: US 2008/0058586 A1
(43) Pub. Date: Mar. 6, 2008

(54) HOOD MEMBER FOR USE WITH AN ENDOSCOPE

(75) Inventor: John A. Karpiel, Winston-Salem, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 11/849,736

(22) Filed: Sep. 4, 2007

Related U.S. Application Data
(60) Provisional application No. 60/842,486, filed on Sep. 5, 2006.

(51) Int. Cl. A61B 1/005

(52) U.S. Cl. 600/104; 600/127

(57) ABSTRACT

The present invention provides apparatus and methods for performing endoscopic mucosal resection and endoscopic submucosal dissection of tissue. In a first embodiment, a hood member having a hood portion and a lever portion is provided. The hood portion is adapted to be disposed over a distal region of an endoscope. A portion of the lever portion is configured to be inserted beneath a section of mucosal tissue having a lesion, and the lever portion is configured to be rotated or otherwise maneuvered to lift the mucosal tissue in an upward direction, thereby facilitating removal of the tissue comprising the lesion. Optionally, a surgeon may advance a needle knife through the endoscope and lever portion to further incise submucosal tissue while the lever portion is disposed beneath the lesion. If desired, a flushing fluid may be provided to a target site during the procedure.

### PRIORITY CLAIM

This invention claims the benefit of priority of U.S. Provisional Application Ser. No. 60/842,486, entitled “Hood Member For Use With an Endoscope,” filed Sep. 5, 2006, the disclosure of which is hereby incorporated by reference in its entirety.

### TECHNICAL FIELD

The present invention relates generally to enhanced apparatus and methods for performing an endoscopic mucosal resection or submucosal dissection of tissue.

### BACKGROUND INFORMATION

Diagnostic and therapeutic gastrointestinal endoscopy are commonly used to gain access to the digestive tract for the purpose of removing tissue. One technique for obtaining tissue for biopsies is an endoscopic mucosectomy procedure, also known as endoscopic mucosal resection (“EMR”). The EMR procedure may be a useful tool for providing a tissue specimen for surgical pathology.

The EMR procedure also may be used for curative purposes to remove sessile benign tumors and intramucosal cancers, and in particular, EMR is a well-accepted treatment for early gastric cancer without lymph node metastasis. During curative removal of a mucosal lesion, it is desirable to perform “en-bloc resection” of the lesion, i.e., removal in one piece. If the lesion is removed in a piecemeal fashion, it is believed that rates of local tumor recurrence may be increased. Further, assessment of fragmented tissue may be more difficult than assessment of unfragmented tissue.

During an EMR procedure, it may be desirable to mark and subsequently resect a portion of tissue surrounding a lesion to ensure that the lesion is completely resected in an en-bloc fashion. In addition to removing the mucosal tissue, a portion of the submucosa also may be removed.

A typical EMR procedure involves identifying the mucosal lesion using an endoscope. The boundaries of the lesion may be marked to facilitate removal. A fluid, such as saline or sodium hyaluronate, may be injected into the submucosal layer just beneath the lesion to help the lesion protrude away from the remaining healthy tissue. A snare may be used to resect the mucosal tissue that includes the lesion. A forceps or snare may be used to grasp and remove the resected tissue via the endoscope.

One reported drawback associated with conventional EMR procedures is that the snaring method tends to yield piecemeal resection of a lesion, which may ruin the histopathologic assessment of the lesion. Further, EMR procedures generally are not recommended for large lesions, e.g., over 2 cm in diameter.

Recently, a technique called endoscopic submucosal dissection (“BESD”) has been developed in which mucosal lesions are removed by the dissection of submucosa under the lesion using an incision device, such as an endoscopic knife. The ESD procedure may facilitate resection of larger lesions and yield improved en-bloc resection, as compared to a conventional EMR procedure.

In view of the drawbacks of current technology, it is desirable to develop apparatus and methods for an EMR or ESD procedure that may efficiently remove mucosal and/or submucosal tissue in unfragmented portions in a relatively short period of time without inducing significant patient trauma.

### SUMMARY

The present invention provides apparatus and methods for performing EMR and ESD procedures. In a first embodiment, the apparatus comprises a hood member comprising a hood portion and a lever portion. The hood portion is adapted to be placed at least partially over a distal region of an endoscope. The lever portion comprises a distal opening, such that one or more medical devices may be advanced through the lever portion to a target site.

In accordance with one aspect, a portion of the lever portion is configured to be inserted beneath a section of mucosal tissue having a lesion during an ESD procedure. The lever portion is configured to be advanced, rotated or otherwise maneuvered to lift the mucosal tissue in an upward direction, thereby facilitating removal of the tissue comprising the lesion.

The hood portion has an interior surface and a hollow lumen formed therein, whereby the lumen of the hood portion is adapted to be placed at least partially over a distal region of an endoscope. The hood portion preferably comprises an elastic member having a first inner diameter in a relaxed state and a larger second diameter in an expanded state. The hood portion may be placed over the endoscope in the expanded state, and is configured to be secured about the endoscope in the relaxed state. The interior surface of the hood portion may comprise an engaging surface comprising a frictional element or an adhesive to facilitate attachment to an exterior surface of the endoscope.

In a preferred method of operation, the endoscope having the hood member attached thereto is delivered towards a target tissue site, and the lever portion of the hood member is disposed adjacent the tissue site. A needle knife may be advanced through a working channel of the endoscope and through the distal opening in the lever portion, and may be used to make markings in the tissue to define the boundaries of the lesion prior to incision of the tissue. In a next step, a needle may be advanced to pierce the mucosal tissue and deliver fluid, such as saline, to the submucosal layer beneath the target tissue site. This fluid injection causes the mucosal tissue having the lesion to bulge outward, i.e., away from the muscularis propria. In a next step, the needle knife may be used to incise the tissue to be removed, e.g., by applying electrical current to the distal tip of the needle knife.

In a next step, the lever portion of the hood member is positioned at least partially beneath the mucosal tissue to be removed. The lever portion then may be gently advanced, rotated and/or rocked in a prying motion, which may facilitate removal or detachment of the incised tissue. If desired, a surgeon may advance the needle knife through the lever portion to further incise submucosal tissue.

In various alternative embodiments, the lever portion may comprise numerous shapes. For example, the lever portion may comprise concave or convex surfaces, a hexagonal distal edge, a pointed distal region, and so forth. Many of the shapes may comprise functional advantages, for example, a concave upper surface of the lever portion may facilitate scooping/dislodging of a lesion that has previously been partially excised by a needle knife.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a perspective view of a first embodiment of a hood member.

FIG. 2 is a side view of the hood member of FIG. 1 disposed over a distal region of an endoscope.

FIGS. 3A-3B are, respectively, a side view and a top view of a method step that may be used in accordance with principles of the present invention.

FIGS. 4A-4B are, respectively, a side view and a top view of another method step that may be used in accordance with principles of the present invention.

FIG. 5 is a side view of a method step that may be used in conjunction with the present invention.

FIG. 6 is a side view of a method step that may be used in conjunction with the present invention.

FIG. 7 is a side view of a method step that may be used in conjunction with the present invention.

FIG. 8 is a side view of a method step that may be used in conjunction with the present invention.

FIGS. 9A-9B are top views illustrating alternative configurations of the hood member of FIGS. 1-8.

FIGS. 10A-10B are side views illustrating further alternative configurations of the hood member of FIGS. 1-8.

FIGS. 11A-11C are end views illustrating further alternative configurations of the hood member of FIGS. 1-8.

### DETAILED DESCRIPTION OF THE PREFERRED EMBODIMENTS

In the present application, the term “proximal” refers to a direction that is generally towards a physician during a medical procedure, while the term “distal” refers to a direction that is generally towards a target site within a patent's anatomy during a medical procedure.

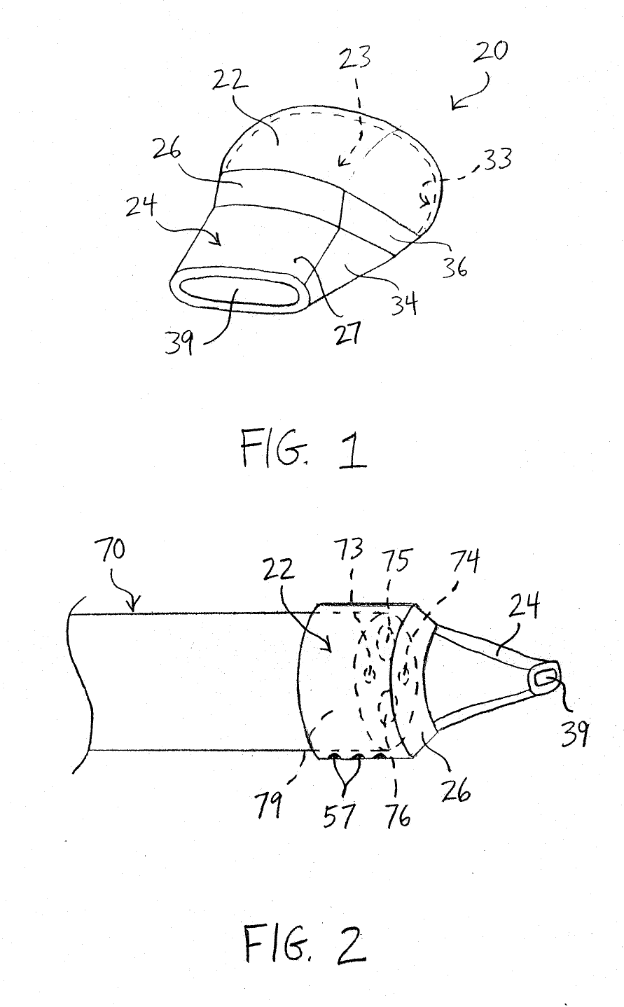

Referring now to FIG. 1, a first embodiment of a hood member of the present invention is shown. In FIG. 1, hood member 20 comprises hood portion 22 and lever portion 24. Hood portion 22 has interior surface 33 and hollow lumen 23 formed therein. Hood portion 22 may comprise a circular, oval or other configuration when viewed from the end (see, e.g., FIGS. 11A-11C below). As will be explained in greater detail below, hood portion 22 is adapted to be at least partially disposed over a distal region of a conventional endoscope, such as endoscope 70 of FIG. 2.

Referring still to FIG. 1, lever portion 24 preferably comprises a shape similar to a flat-head screwdriver. However, lever portion 24 may have many different shapes, for example, as shown with respect to FIGS. 9-11 (discussed below). In the embodiment of FIG. 1, lever portion 24 has substantially flat upper region 27 and substantially flat side region 34. An opposing lower region and side region (not shown) form a four-plane lever shape. Opening 39 is formed at the distal edge of lever portion 24. Opening 39 is adapted to receive various medical devices, such as a needle knife or injection needle, as will be explained in greater detail below.

Viewing portion 26 preferably is disposed between hood portion 22 and lever portion 24. Viewing portion 26 may comprise a more pronounced taper compared to lever portion 24, i.e., viewing portion 26 may be more orthogonal to a longitudinal axis of hood member 20, as depicted in FIG. 1. Both lever portion 24 and viewing portion 26 comprise a biocompatible, transparent material. Therefore, when hood portion 22 is disposed over endoscope 70, as shown in FIG. 2, a physician may have a substantially unobstructed view of the anatomical features and medical components in front of the endoscope.

As shown in FIG. 2, distal region 79 of endoscope 70 may comprise optical elements 73 and 74, which may employ fiber optic components for illuminating and capturing an image distal to the endoscope. Further, endoscope 70 preferably comprises auxiliary lumen 75 and working channel 76. As noted above, working channel 76 preferably is sized to accommodate an array of components for performing an EMR or ESD procedure, such as a needle, needle knife, forceps, snare, and the like. It will be apparent to one skilled in the art that while one auxiliary lumen 75 and one working channel 76 are shown, endoscope 70 may comprises any number of lumens/channels to achieve the objects of the present invention.

In one embodiment, hood portion 22 comprises an elastic member, such that lumen 23 has a first inner diameter in a relaxed state, but when expanded radially outward, lumen 23 may assume a second, slightly larger inner diameter. Lumen 23 is sized to be disposed about an exterior surface of endoscope 70, as shown in FIG. 2. If hood portion 22 is elastic, it may be sized such that its inner diameter in the relaxed state is slightly smaller than an exterior diameter of endoscope 70, but its inner diameter in the expanded state is slightly larger than the exterior diameter of endoscope 70. Therefore, hood portion 22 may be elastically expanded to fit over distal region 79 of endoscope 70. Once in place, hood portion 22 in the relaxed state will be securely engaged around the exterior surface of endoscope 70 using a frictional fit, as shown in FIG. 2. Interior surface 33 of hood portion 22 may comprise a texture or material, such as rubber, to increase the frictional fit with the exterior surface of endoscope 70. Optionally, friction members 57 of FIG. 2 may be employed to reduce the likelihood of movement once hood portion 22 is secured about endoscope 70. Alternatively, an adhesive (not shown) may be placed on an exterior surface of endoscope 70 and/or interior surface 33 of hood portion 22 to promote a secure attachment of the components.

If desired, an external securing means may be employed to secure a proximal region of hood portion 22 directly to the exterior surface of endoscope 70. For example, an adhesive tape, heat-shrink tubing, one or more tie-down bands, cable-ties, and the like may be employed at an interface between hood portion 22 and endoscope 70, thereby securing the hood member over the distal region of the endoscope.

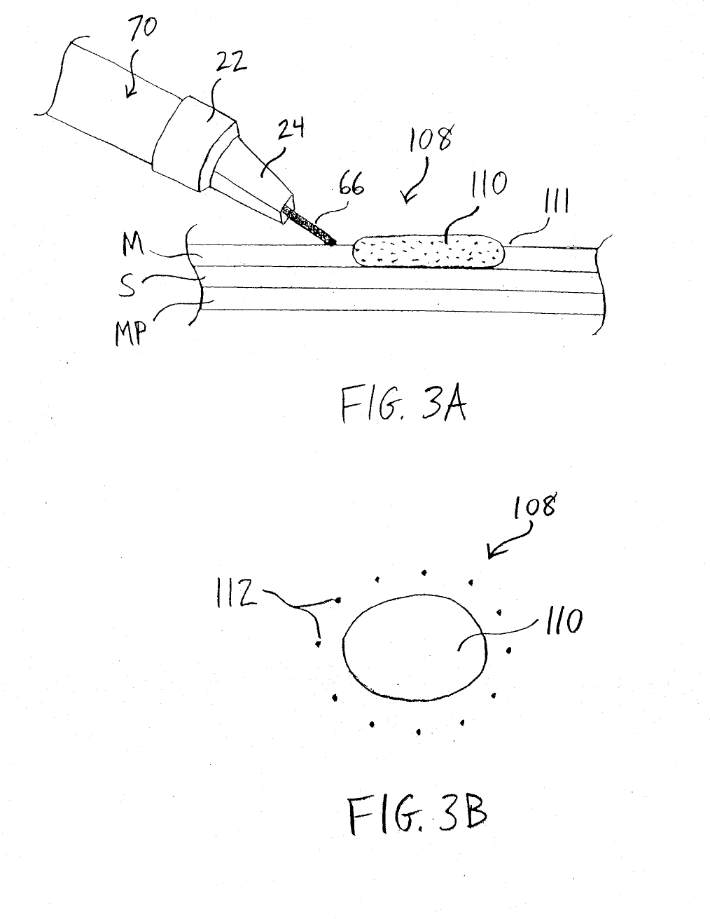

Referring now to FIGS. 3-8, a method for performing an EMR or ESD procedure in accordance with principles of the present invention is described. In a first step, endoscope 70 is maneuvered towards a target tissue site 108 using endoscopy techniques that are known in the art. For example, the device may be maneuvered into a patient's mouth, down through the esophagus and duodenum, and towards the target tissue site 108. Target tissue site 108 may comprise lesion 110, e.g., indicative of gastric cancer, which is fully or partially confined within mucosal tissue layer M. Beneath musoca M, submusoca S and muscularis propria MP are present, as shown in FIG. 3A.

Once endoscope 70 is positioned adjacent target tissue site 108, a physician will examine whether incision markings are needed to define the boundaries of target tissue site 108, If the margins 111 of target tissue site 108 are not readily discernible, needle knife 66 may be loaded through auxiliary lumen 75 or working channel 76 of endoscope 70. Needle knife 66 may then be advanced distal to endoscope 70, through lever portion 24 and through distal opening 39 of hood member 20. Needle knife 66 then may be used to engage the target tissue and create markings 112 around margins 111 of target tissue site 108, as depicted in FIG. 3B. High frequency current may be applied to the needle knife tip to create the markings. Such methods for creating markings are well known to those of ordinary skill in the art. Alternatively, markings 112 may be omitted where target tissue site 108 can readily be distinguished from tissue not intended to be cut.

In accordance with one aspect, the tapered shape of viewing portion 26 and lever portion 24 facilitates distal advancement of medical devices, such as needle knife 66, towards a target site after the device exits distal to endoscope 70. Specifically, viewing portion 26 and lever portion 24 will guide needle knife 66 and other devices through opening 39 and to the desired site. Since viewing portion 26 and lever portion 24 are transparent, a physician may easily track the advancement of needle knife 66 via optical elements 73 and 74.

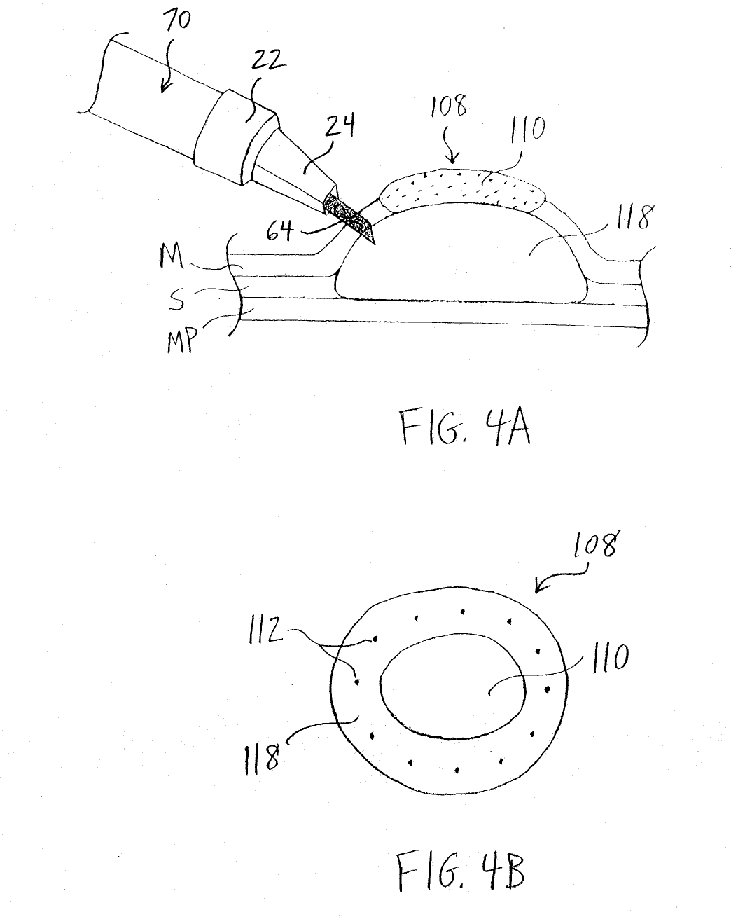

Referring now to FIGS. 4A-4B, in a next step, the targeted mucosal tissue may be lifted with respect to muscularis propria MP to facilitate removal of lesion 110. Protrusion of target tissue site 108 may be achieved by injecting a fluid, such as physiological saline solution or sodium hyaluronate, through needle 64. Needle 64 and needle knife 66 may be disposed and advanced through the same or different lumens of endoscope 70. For example, needle 64 may be disposed within auxiliary lumen 75, while needle knife 66 is advanced through working channel 76. Alternatively, needle knife 66 may be disposed within a hollow interior region of needle 64, and fluid may be injected through needle 64 such that it flows around needle knife 66.

As shown in FIG. 4A, the fluid injection into submucosa S lifts target tissue site 108 from the underlying muscularis propria MP, thereby forming fluid pocket 118 in submucosal layer S. Fluid pocket 118 is shown from an elevated view in FIG. 4B. By elevating target tissue site 108 having lesion 110, a subsequent excision of lesion 110 is facilitated, as explained in greater detail below. Elevation of the target tissue facilitates removal of the lesion during an endoscopic mucosal resection procedure. The ability to remove the abnormal tissue without cutting into it enables a more accurate assessment of the tissue than would otherwise be possible if sampling a fragmented tissue sample. Furthermore, fragmented resection of early cancers may lead to a higher rate of local tumor recurrence.

Referring now to FIG. 5, after target tissue site 108 has been sufficiently elevated, the process of creating a mucosal incision may begin. Needle 64 may be retracted proximally to be confined within endoscope 70, and needle knife 66 may be advanced distally beyond lever portion 24 and through opening 39, as depicted in FIG. 5.

The mucosal incision may be made circumferentially around lesion 110 using needle knife 66, as depicted in FIG. 5. An electrosurgical generator (not shown) may be coupled to needle knife 66 to provide an electrical energy sufficient to incise the tissue. The incision preferably is performed at a predetermined distance into submucosa S, and at a predetermined angle with respect to muscularis propria MP.

Needle knife 66 may be fabricated from any electrically conductive material, including stainless steel. Alternatively, it may be fabricated from a shape memory alloy such as nitinol, as described in co-pending U.S. patent application Ser. No. 11/729,402, filed Mar. 28, 2007. Optionally, needle knife 66 may comprise a non-conductive portion at its tip, such as a hollow or ceramic region, which helps prevent the needle knife from cutting too far into tissue. Other safety mechanisms will be apparent to one skilled in the art.

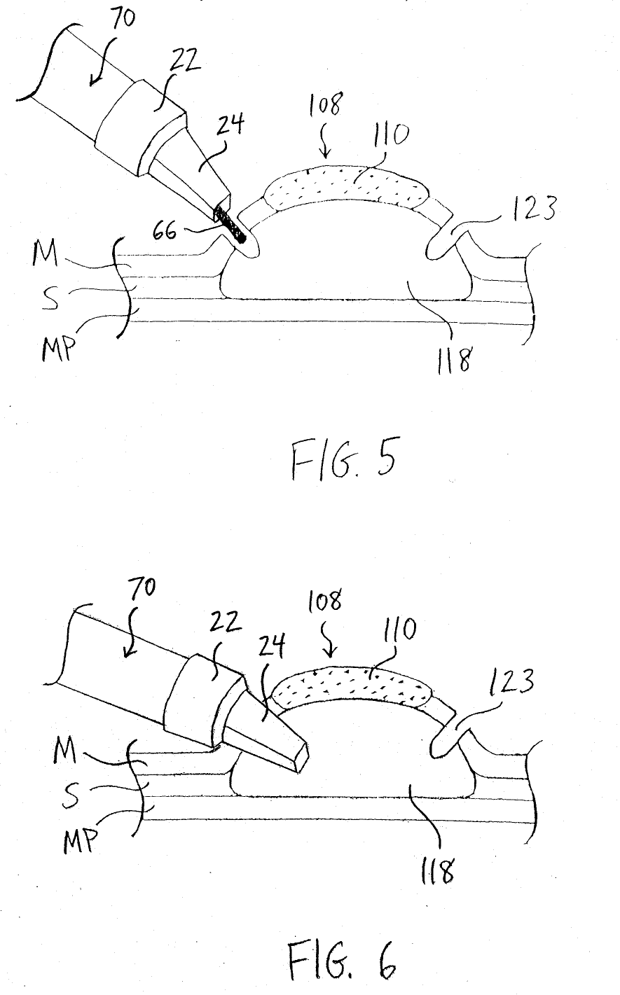

Referring now to FIG. 6, after target tissue 108 has been partially or completely incised, needle knife 66 is retracted to withdraw the distal end of the needle knife completely into endoscope 70. In a next step, endoscope 70 is advanced in a distal direction and at an angle such that lever portion 24 at least partially pierces through mucosa M and into fluid pocket 118 within submucosa S. At this time, endoscope 70 preferably is positioned such that a portion of lever portion 24 is disposed beneath a portion of target tissue site 108, as shown in FIG. 6. Hood portion 22 may abut mucosa M, as depicted in FIG. 6, or alternatively may be disposed proximal or distal to the mucosal wall.

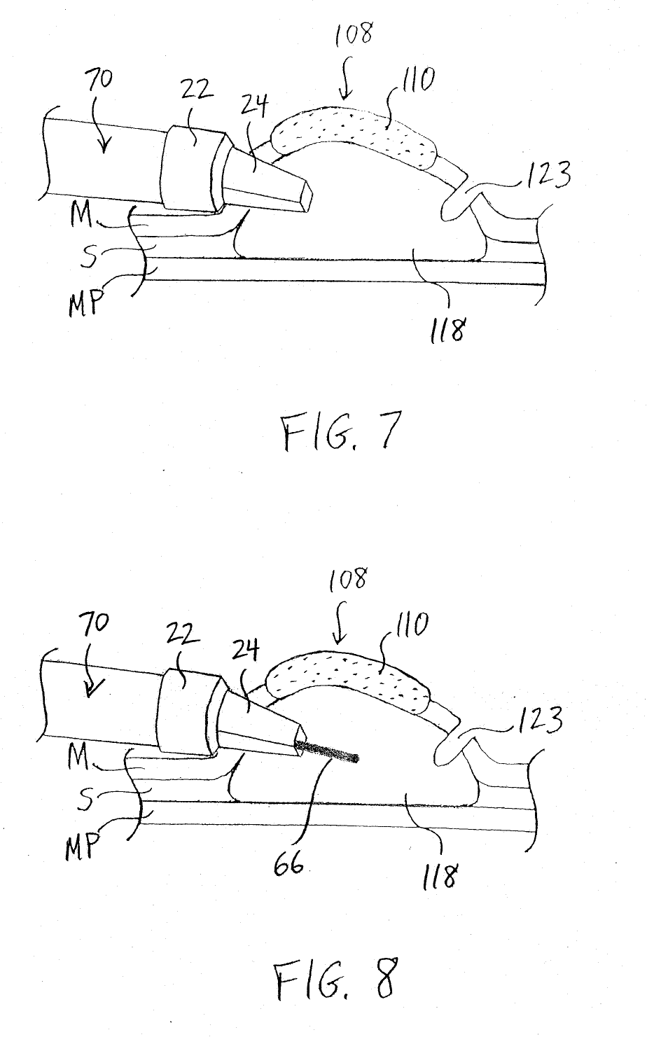

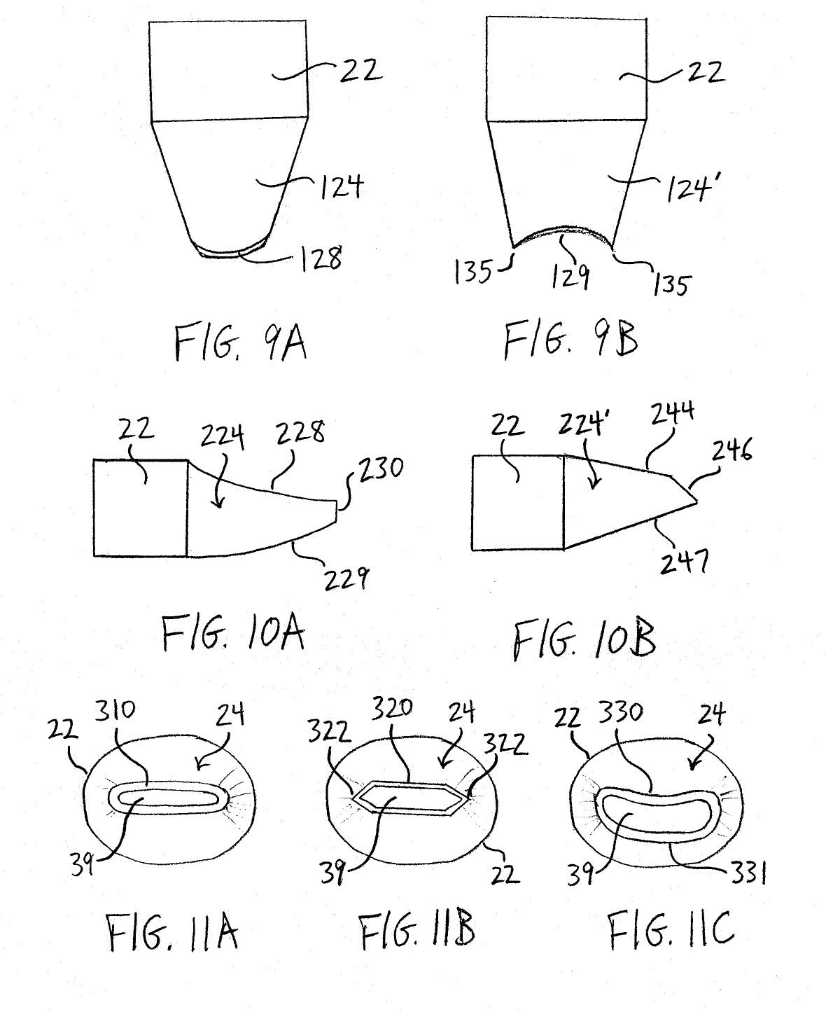

Upon proper positioning, endoscope 70 is maneuvered such that lever portion 24 lifts up target tissue site 108 from beneath it, thereby facilitating resection of lesion 110. In particular, distal region 79 of endoscope 70 is gently advanced, rocked and/or rotated at a predetermined angle with respect to muscularis propria MP, causing endoscope 70 to be more parallel to muscularis propria MP layer, as shown in FIG. 7. Such prying movement of lever portion 24 may help dislodge the mucosal portion of target tissue site 108 away from submucosa S. In the process, portions of submucosa S also may be drawn away from muscularis propria MP. While lever portion 24 is depicted as being disposed under a relatively small portion of lesion 110 in FIGS. 7-8, it will be apparent that the lever portion may be advanced further beneath the lesion prior to being gently rotated or rocked.

Referring now to FIG. 8, while lever portion 24 pries target tissue site 108 away from submucosa S and/or muscularis propria MP, needle knife 66 optionally may be advanced beyond the distal tip of lever portion 24, thereby dissecting submucosal tissue from within fluid pocket 118. Therefore, in addition to the mucosal resection procedure performed in FIG. 5, a submucosal dissection may be achieved in FIG. 8 to facilitate “en-bloc” removal of target tissue site 108. It should be appreciated that while a needle knife 66 is depicted as the cutting device in FIG. 8, other electrified or mechanical endoscopic cutting instruments may be employed, such as a scalpel and the like.

Once the incised target tissue is sufficiently separated from its surrounding tissue, needle knife 66 may be withdrawn and endoscope 70 may be retracted to remove lever portion 24 from beneath the target tissue. A retrieval device, such as a snare or forceps (not shown), ten may be advanced through auxiliary lumen 75 or working lumen 76 to subsequently remove incised target tissue 108, which includes lesion 110. The endoscope then may be removed from the patient to complete the procedure.

Advantageously, by employing a hood member in conjunction with a conventional endoscope, a surgeon may selectively maneuver lever portion 24 of the hood member beneath target tissue site 108 to help dislodge the incised mucosal tissue. Further, as noted above, the submucosal dissection techniques described herein may promote “en-bloc” removal of lesion 110 to improve subsequent pathological assessment of the lesion.

If desired, flushing fluid may be provided to the target tissue site at any time during the EMR or ESD procedure. For example, the flushing fluid may be delivered through auxiliary lumen 75 or working channel 76, and may be delivered around needle 64 and/or needle knife 66, as described in co-pending U.S. patent application Ser. No. 11/747,570, filed May 11, 2007, which is hereby incorporated by reference in its entirety.

Referring now to FIGS. 9-11, various alternative configurations of the hood member of the present invention are shown. In FIG. 9A, a top view of alternative lever portion 124 is shown. Lever portion 124 comprises an edge that is tongue-shaped or convex. Therefore, as lever portion 124 is advanced distally, the convex shape may facilitate insertion of the distal edge of lever portion 124 beneath target tissue site 108. By contrast, as shown in FIG. 9B, alternative lever portion 124′ comprises a concave shape, which forms two pointed edges 135. These edges 135 may facilitate separation of target tissue along the margins.

In FIG. 10A, a side view of an alternative lever portion 224 is shown. Lever portion 224 comprises curved upper surface 228, curved lower surface 229, and flat distal opening 230. Curved upper and lower surfaces 228 and 229 are designed to facilitate distal advancement of lever portion 224 beneath a target tissue site. Further, the design of curved upper surface 228 may help scoop out or otherwise dislodge a portion of a lesion, as explained above. By contrast, in FIG. 10B, alternative lever portion 224′ comprises upper surface 244, lower surface 247, and pointed edge 246, which may facilitate advancement of the lever portion 224′ beneath target tissue site 108 and/or resection of lesion 110.

In FIG. 11A, opening 39 of lever portion 24 is surrounded by a substantially elliptical edge 310. In FIG. 11B, opening 39 is surrounded by a hexagonal edge. The hexagonal edge comprises relatively long upper and lower portions 320, along with two opposing pointed edges 322. Finally, in FIG. 11C, opening 39 is surrounded by a concave upper edge 330 and a substantially similar concave lower edge 331. In this embodiment, concave upper edge 330 may help scoop out or otherwise dislodge a portion of a lesion, after lever portion 24 has been disposed beneath the target tissue site, as explained above.

It will be appreciated that the apparatus and methods described hereinabove may be used to treat various types of lesions, e.g., large superficial tumors and intraepithelial neoplasms, in virtually any body cavity, such as the stomach, esophagus and colon.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

I claim:

1. Apparatus suitable for performing mucosal resection of tissue, the apparatus comprising:
a hood portion having an interior surface and a lumen formed therein, the lumen being adapted to be at least partially disposed over a distal region of an endoscope; and
a lever portion coupled to the hood portion, the lever portion having a distal opening configured to permit the advancement of one or more medical components,
wherein a portion of the lever portion is configured to be inserted beneath a section of mucosal tissue to lift a targeted mucosal layer of the tissue in an upward direction to facilitate removal of the tissue.

2. The apparatus of claim 1 wherein the lever portion comprises a transparent material.

3. The apparatus of claim 1 wherein the interior surface of the hood portion comprises an engaging surface comprising a frictional element or an adhesive to facilitate attachment to an exterior surface of the endoscope.

4. The apparatus of claim 1 wherein the hood portion comprises an elastic member, wherein the lumen has a first inner diameter in a relaxed state and a larger second diameter in an expanded state, wherein the hood portion is adapted to be placed over the endoscope in the expanded state.

5. The apparatus of claim 4 wherein the first inner diameter of the hood portion in the relaxed state is smaller than an outer diameter of the endoscope to permit the hood portion to be elastically secured about an exterior surface of the endoscope.

6. The apparatus of claim 1 further comprising a viewing portion coupled between the hood portion and the lever portion, wherein the viewing portion comprises a transparent material.

7. The apparatus of claim 1 wherein the lever portion comprises a curved upper surface to facilitate dislodging a mucosal layer of the tissue when the lever portion is disposed at least partially beneath the mucosal layer of the tissue.

8. The apparatus of claim 1 wherein the distal opening of the lever portion comprises a pointed edge.

9. A method suitable for performing mucosal resection of tissue, the method comprising:
providing a hood portion having an interior surface and a lumen formed therein, and providing a lever portion coupled to the hood portion, the lever portion having a distal opening configured to permit the advancement of one or more medical components;
disposing the lumen of the hood portion at least partially over a distal region of an endoscope and securely coupling the hood portion to the distal region of the endoscope;
inserting a portion of the lever portion beneath a section of mucosal tissue; and
maneuvering the lever portion to lift a targeted mucosal layer of the tissue in an upward direction to facilitate removal of the tissue.

10. The method of claim 9 wherein the lever portion comprises a curved upper surface, the method further comprising using the lever portion to facilitate dislodging a mucosal layer of the tissue when the lever portion is disposed at least partially beneath the mucosal layer of the tissue.

11. The method of claim 9 further comprising using a needle to inject fluid into a submucosal layer of the tissue to raise a portion of the targeted mucosal layer of the tissue in an upward direction.

12. The method of claim 9 further comprising delivering a needle knife through the endoscope and resecting the targeted mucosal layer of the tissue, wherein sufficient tissue is resected such that a portion of the lever portion can be inserted beneath the mucosal tissue.

13. Apparatus suitable for performing mucosal resection of tissue, the apparatus comprising:
a hood portion having an interior surface and a lumen formed therein, the lumen being adapted to be at least partially disposed over a distal region of an endoscope;
a lever portion coupled to the hood portion, the lever portion having a distal opening configured to permit the advancement of one or more medical components; and
a transparent viewing portion coupled between the hood portion and the lever portion,
wherein at least the distal opening of the lever portion is configured to be inserted beneath a section of mucosal tissue, and the lever portion comprises at least one region configured to lift a targeted mucosal layer of the tissue in an upward direction to facilitate removal of the tissue.

14. The apparatus of claim 13 wherein the lever portion comprises a curved upper surface to facilitate dislodging a mucosal layer of the tissue when the lever portion is disposed at least partially beneath the mucosal layer of the tissue.

15. The apparatus of claim 13 wherein the distal opening of the lever portion comprises a pointed edge.

16. The apparatus of claim 13 wherein the interior surface of the hood portion comprises an engaging surface comprising a frictional element or an adhesive to facilitate attachment to an exterior surface of the endoscope.

17. The apparatus of claim 13 wherein the hood portion comprises an elastic member, wherein the lumen has a first inner diameter in a relaxed state and a larger second diameter in an expanded state, wherein the hood portion is adapted to be placed over the endoscope in the expanded state.

18. The apparatus of claim 13 further comprising a needle configured to pierce through mucosal tissue to deliver fluid to submucosal tissue, wherein the needle has an outer diameter that is configured to be disposed through a working channel or an auxiliary lumen of the endoscope and the distal opening of the lever portion.

19. The apparatus of claim 13 further comprising a needle knife configured to be inserted through a working channel or an auxiliary lumen of the endoscope and the distal opening of the lever portion.

20. The apparatus of claim 13 further comprising an endoscope having proximal and distal regions, wherein the hood portion is disposed at least partially over the distal region of the endoscope.
